(19) United States
(12) Patent Application Publication
(10) Pub. No.: US 2016/0184545 A1
(43) Pub. Date: Jun. 30, 2016

(54) AIRWAY ADAPTER AND METHOD FOR MOLDING THE AIRWAY ADAPTER

(71) Applicant: GENERAL ELECTRIC COMPANY, Schenectady, NY (US)

(72) Inventor: Jani Mikael Kauppi, Helsinki (FI)

(21) Appl. No.: 14/860,237

(22) Filed: Sep. 21, 2015

(30) Foreign Application Priority Data
Dec. 31, 2014 (GB) 1423385.2

(51) Int. Cl. A61M 16/08; B29C 45/26

(52) U.S. Cl. CPC A61M 16/0816; B29C 45/26; A61M 2207/10; A61M 2205/02; B29C 2045/2695; B29K 2023/06; B29K 2023/12

(57) ABSTRACT

A method for molding an airway adapter, the airway adapter comprising a flow path for fluid and integral windows to allow a measurement path to pass through the flow path, the method comprises injection molding the airway adapter in a single shot molding step. During the single shot molding step, the windows are initially thicker than their final thickness and reduced to their final thickness by moving pins that press the windows to their final thickness. An airway adapter comprises a flow path for fluid and windows for a measurement path through the flow path. The windows may be located on opposite sides of the airway adapter; the windows may be integral with the airway adapter forming one solid part of the airway adapter jointlessly and continuously; and the windows may have a thickness of less than 0.2 mm.

### TECHNICAL FIELD

The present disclosure relates to an airway adapter. More particularly, the present disclosure relates to an airway adapter and a method for molding the airway adapter.

### BACKGROUND

During anaesthesia or in critical care, patients are often mechanically ventilated instead of breathing spontaneously. The patient is connected to a patient circuit of a ventilator or an anaesthesia machine by intubation or non-intubation. Intubation is when an endotracheal tube is inserted into a trachea so that a gas can flow through it into and out of a lung. During intubation the breathing circuit typically comprises the endotracheal tube, a Y-piece where inspiratory and expiratory tubes from the ventilator come together, as well as the ventilator. Non-intubation is when the endotracheal tube is replaced with a breathing mask, which covers nasal and/or oral cavities so that a gas can flow through it into and out of a lung.

A mainstream gas analyzer normally comprises an airway adapter, which connects to a breathing gas measuring sensor. The airway adapter is placed into the breathing circuit between the endotracheal tube or the breathing mask and the breathing circuit Y-piece to allow the breathing gas to flow through the airway adapter to be measured. Usually the airway adapter is replaced between the patients, to prevent cross contamination between the patients, and normally at least every day to prevent bacteria to grow over unacceptable levels increasing infection risk.

A way to measure gas concentration of some gases, especially carbon dioxide and commonly used volatile anaesthetics, is based on the gas absorption of non-dispersive infrared radiation (IR) at measured gas specific wave lengths. A problem in IR gas analyzers, especially in mainstream gas analyzers is the measurement signal drift or change during or between the measurements. The infrared radiation generated by the IR source traverses through the airway adapter and its optical path comprising different boundary surfaces such as optical windows of a gas sampling cell of the airway adapter before reaching the measuring detector. The optical path of a gas sampling cell changes when the airway adapter is replaced. There are optical differences between the gas sampling cells due to manufacturing tolerances, material differences and optical path deformation with time, which cause gas analyzer measurement signal differences or error between airway adapters. The measurement signal error can include offset error that can be seen as inspired/expired gas concentration signal offset or gain error that can be seen as an increasing error proportional to the measured gas concentration.

It is a challenge in existing mainstream gas analyzers to take into account the optical path differences between airway adapters, e.g. when an airway adapter is replaced. For example, a change in material and thickness of the optical windows causes changes in spectral transmission and changes in the diameter of optical window openings cause changes in transmission, which can be seen as offset error. Manufacturing tolerances related to an optical path length, for example due to individual part tolerances and assembly accuracy tolerances, cause differences in gas absorption that can be seen as gain error. In addition, components such as an IR-source and measuring detectors may drift at the same time increasing all these errors.

The ventilation must meet the patient's needs for a correct exchange and administration of oxygen (O2), carbon dioxide (CO2), nitrous oxide (N2O), anaesthetic agents and other gases. Anaesthesiologists, respiratory therapists and other qualified clinicians use their professional skills to set ventilation parameters to optimally meet needs of the patient. The ventilation is often monitored with a respiratory monitor by measuring essentially in real time concentrations of oxygen, carbon dioxide, nitrous oxide and anaesthetic agent in a breathing gas.

Each time an airway adapter is replaced in a gas analyser, the gas analyser must be zeroed and may have to be calibrated. This generates additional work for the hospital personnel and reduces the time for taking care of patients. Zeroing and calibration routines are complex and time consuming. As the care situations they are used in are often demanding, there is a high probability of zeroing and calibration errors. If the zeroing or calibration routines are disregarded the sensor may likely show wrong values generating a risk for a patient.

Leakage is one possible cause of problems in an airway adapter. An adapter that is assembled from multiple different components may therefore have more possible leakage paths. Even small leakage rates can cause significant problems, for example with leakage occurring in the seam between the window and the adapter body. A leakage may lead to a further absorption between the sensor and the airway adapter causing incorrect reading. For example, a leak with a high concentration of CO2 can cause additional absorption and therefore lead to a higher CO2 concentration reading. Adapter windows are separate parts that are assembled into a body part. There might be also additional components included in the window assemblies. Each seam can be a potential leakage path. For example inexpensive, tough and highly chemically resistant materials with simple microstructure like polypropylene and polyethylene have poor gluing, heat sealing and welding characteristics.

Cost is another important variable. Disposable adapters are changed frequently, therefore creating significant cost to hospitals. Assembly work and the complexity of an assembly define a big part of the cost. A more complex assembly structure may also have increased number of potential leakage paths therefore creating higher probability of failure and lower yield—that transforms into increased product cost. The higher probability of failure can also lead to more intense testing during production. That again increases the part cost.

One known method of window attachment is to glue the window to the adapter body. That usually leads to a small backstep behind the window inside the adapter. That can lead to problems with moisture. Moisture is very often present in the ventilation circuit. Moisture can come from the patient, active humidification and other common moisture sources within the system. The backstep tends to capture the moisture droplets very effectively, increasing the IR absorbance through the measurement path and distorting the measurement value.

One known method is to injection mold the windows as separate parts and then to attach the windows to a separate body part. Such injection molded windows must be thick and must be glued to the adapter body. This leads to joints between the window and the adapter body. This is a problem because leakage may occur in the joints.

Injection molding is a process capable of creating thin walled parts, but it has technical limitations with respect to how thin the wall can be made. For example, a window thickness of less than 0.2 mm can only be made for a very small surface area for the thin walled section, making the technique not suitable for adapter windows. In other words, a window manufactured by injection molding for an airway adapter will have no good transmission and an uneven thickness.

Problems also occur if the thin walled window sections are combined with a larger part comprising thick wall sections. Problems can also be expected to increase as the part gets bigger. Thicker sections fill more easily and therefore the flow of material is more difficult to direct into the window area. In other words, the injection molded windows should be thicker if they are integrated into a bigger part having also thicker wall sections, as for an airway adapter. Typically the window area, radius or width and length, are limited by mechanical requirements to a minimum of a few millimetres. This means that, if the windows were integrated into an adapter body or similar bigger part and the part was manufactured using single shot injection molding, the window thickness would have to be even larger than, for example, about 0.2 mm of a separately injection molded window.

Limiting factors are for example the flow characteristics of the selected material. The flow tends to flow along the easiest path and tends to avoid going into the more narrow areas where the narrow flow path tends to restrict the material flow. The flow also cools down as the distance from the injection point increases. This makes the molten material flow worse than when the material is hotter. Thus, there is a technical problem as the window area increases or the window thickness decreases. These problems lead to leakage of the injection molded airway adapter. When manufacturing a product that has similar features to an airway adapter window by injection molding a part of the product must be at least 0.2 mm thick for manufacturing reasons. If a thin walled section in a part becomes larger, the injection molding process becomes more difficult and at some point impossible. This happens if a wall thickness is not increased to compensate the larger area.

Using multi shot injection molding does not improve the problems discussed above. Multi shot injection molding causes the different shots to solidify at different times. This causes joints and discontinuities and seams between features that lead to leakage. The process also restricts the minimum thickness of the windows in the same way as the one shot process and as described above.

It is further a problem to find an economical and at the same time technical solution to manufacture an airway adapter. Many additional restrictions must be considered because of regulations and health and safety aspects.

### SUMMARY

The present disclosure is directed to a method for molding an airway adapter and an airway adapter. This can be achieved by the features as defined by the independent claims. Further enhancements are characterized in the dependent claims.

In one embodiment, the present disclosure is directed to a method for molding an airway adapter. The airway adapter comprises a flow path for fluid and integral windows to allow a measurement path to pass through the flow path. The method comprises injection molding the airway adapter in a single shot molding step. Wherein during the single shot molding step, the windows are initially thicker than their final thickness and reduced to their final thickness by moving pins that press the windows to their final thickness.

According to at least one embodiment, each window may have one moving pin. According to one embodiment, each of the pins reduce the final window thickness to less than 0.2 mm, preferably less than 0.15 mm, preferably less than 0.1 mm, preferably less than 0.05 mm. The pins may move a distance less than 1 mm, preferably less than 0.4 mm, preferably less than 0.2 mm. The pins may move a distance that is the wall thickness surrounding the windows less the window thickness. The two windows may be opposite each other. The windows may be facing the flow path perpendicular. The window may have a diameter of 2 mm to 20 mm, preferably a diameter of 2 mm to 10 mm. The window may have a diameter less then 10 mm, preferably less than 7 mm, preferably less than 5 mm, preferably 4, 3, or 2 mm.

According to at least one embodiment, the relationship between the initial thickness and the final thickness may be about thirty to one, twenty to one, or fifteen to one, or less. According to one embodiment, a wall thickness surrounding the windows may be 30 times, preferably 20 times, preferably 15 times that of the windows thickness 32. Polypropylene or polyethylene may be used for injection molding the airway adapter. Other polymers may also be used. The pins dimensions and/or shape may decide the final size and/or shape of the windows.

In one embodiment, the present disclosure is directed to an airway adapter comprising a flow path for fluid and windows for a measurement path through the flow path, the windows being located on opposite sides of the airway adapter. The windows are integral with the airway adapter forming one solid part of the airway adapter jointlessly and continuously, and the windows having a thickness of less than 0.2 mm.

According to at least one embodiment, the window may have a thickness of less than 0.2 mm, preferably less than 0.15 mm, preferably less than 0.1 mm, preferably 0.05 mm, or less. The window and the airway adapter may be made of one and the same material. The airway adapter may be made entirely out of polypropylene or polyethylene. The window may have a diameter of 2 mm to 20 mm, preferably a diameter of 2 mm to 10 mm. The window may have a diameter less then 10 mm, preferably less than 7 mm, preferably less than 5 mm, preferably 4, 3, or 2 mm.

At least one of the above embodiments provides one or more solutions to the problems and disadvantages with the background art. At least one embodiment has as a technical effect that a very inexpensive complete airway adapter can be manufactured in a short time and of a very good and consistent quality, for example no leakage and high transmission properties and with consistent window to window distance. Other technical advantages of the present disclosure will be readily apparent to one skilled in the art from the following description and claims. Various embodiments of the present application obtain only a subset of the advantages set forth. No one advantage is critical to the embodiments. Any embodiment may be technically combined with any other embodiment(s).

### BRIEF DESCRIPTION OF THE DRAWINGS

The accompanying drawings illustrate presently exemplary embodiments of the disclosure and serve to explain, by way of example, the principles of the disclosure.

FIG. 1 is a diagrammatic illustration of an airway adapter according to an exemplary embodiment of the disclosure;

FIG. 2 is a diagrammatic illustration of a cross section of an airway adapter according to an exemplary embodiment of the disclosure;

FIG. 3 is a diagrammatic illustration of a cross section of an airway adapter according to an exemplary embodiment of the disclosure;

FIG. 4 is a diagrammatic illustration of a window from FIG. 3 according to an exemplary embodiment of the disclosure; and

FIG. 5 is a diagrammatic illustration of a cross section of a mold according to an exemplary embodiment of the disclosure.

### DETAILED DESCRIPTION

One embodiment discloses a method for molding an airway adapter 10. The airway adapter comprises a flow path 20 for fluid and at least two integral windows 30 to allow a measurement path to pass through the flow path 20. The method comprises injection molding the airway adapter 10 in a single shot molding step, wherein during the single shot molding step, the windows 30 are initially thicker than their final thickness and reduced to their final thickness by moving pins 200 that press, coins, the windows 30 to their final thickness. The flow path 20 and the measurement path may be perpendicular to each other. In other words, the windows face perpendicular to the flow path 20 and the windows axes face perpendicular to the flow path axis. Perpendicular in this context may be substantially perpendicular, because a few degrees variation may be acceptable. Substantially may mean for example less than five degrees, preferably less than 2 degrees, preferably less than 1 degree and optimally about zero degrees. Window thickness can be non-uniform, however uniform thickness is one preferred solution. Non-uniform window thickness can result for example from a draft that may be used on the adapter flow path surfaces to for example improve manufacturability or from an angled pin front surface. The draft angle can be for example less than five degrees, preferably less than two degrees, more preferably less than one degree. Optimally there is no draft on the flow path surfaces. The moving pins may for example have an angled front surface that may have similar angles than the said drafted surfaces. The non-uniform thickness and the changes of the axis angles, and their different combinations, may lead to refraction of light, but with the said angle limitations it is acceptable with regards to the measurement accuracy. The airway adapter may have the measurement path through the flow path that is used in respiratory measurements. The measurement path may be constructed from two windows located at opposite sides of the adapter cross sectional perimeter. It may be at least two windows 30 opposite each other allowing a measurement beam to pass through the windows 30 and thereby passing through the airway adapter and the flow path 20 flowing through it. However, more than two windows may be possible, for example four windows for two measurement paths.

According to this embodiment, the complete airway adapter is manufactured by injection molding, including all details such as windows. The airway adapter may be one single piece. The airway adapter may not consist out of anything else than the airway adapter deriving directly from the one single shot injection molding. The airway adapter may need no assembling. According to at least one embodiment, the whole and complete airway adapter can be made during a one single shot molding step, because the windows are manufactured by using moving core pins 200. The windows may thus be manufactured during a single shot injection molding process together with the adapter body using moving core pins that press the windows to their final thickness slightly after the material has filled the area under the pins. The one and the same resin, material, may be used for the whole airway adapter. The airway adapter has thereby no joints or seams or similar. The windows 30 may be integrated into the body of the airway adapter forming one continuous solid part having no joints, seams or other similar imperfections and discontinuities of material. This has as a technical effect that a very inexpensive complete airway adapter can be manufactured in a short time and of a very good and consistent quality, for example no leakage and high transmission properties and with consistent window to window distance.

According to one embodiment, it is preferred to inject the resin into the mold with equal shortest distance to each window. In this way a good flow of resin reaches the windows. The mold comprises two components, see FIG. 5, a first mold half 110 and a second mold half 120. A cavity 11 in the mold is for filling with resin to mold the airway adapter. The cavity 11 is the part of the mold for the airway adapter. The cavity 11 comprises a mold cavity 31 for the windows. The cavity 31 is the part of the mold for windows. It is this cavity 31 that lies in front of the heads of the pins and that the pins 200 press to the final thickness of the windows 30. Hereby the final diameter of the windows 30 is also molded. A material, for example plastic resin such as PP and/or PE, enters the mold through a gate 130 in the injection mold. The mold gate 130 for injecting the resin into the mold may be situated with equal distance to each window cavity 31 or any place where the fill can be ensured. The mold gate 130 for injecting the resin into the mold may be situated with equal shortest distance to each window cavity 31. Multiple gates may be used to reduce distance and ensure filling.

According to at least one embodiment, confidence of fill can be increased around a thin walled window section by injecting the material first into a bigger mold cavity 11 that enables the material to flow fully into the mold around the window cavity 31. The window cavity 31 may be of a suitable dimension to ensure filling. Shortly thereafter, the still molten material is pressed thinner using, for example, a moving core pin 200. The pin 200 presses, coins, some of the molten material away so that a thin layer is made and this layer becomes the window. The force can be applied to the pin 200, for example, by pneumatic or hydraulic systems. The pin 200 can be, for example, the same shape as the window. The diameter of the pin 200 and therefore the window 30 may be 2 mm to 20 mm, preferably a diameter of 2 mm to 10 mm. The window may have a diameter less then 10 mm, preferably less than 7 mm, preferably less than 5 mm, preferably 4, 3, or 2 mm. The final window thickness 32 may be, after the pressing of the pins 200, less than 0.2 mm, preferably less than 0.15 mm, preferably less than 0.1 mm, and optimally around 0.05 mm. The dimension of the window cavity 31, before the pins move, can be, for example, the same as the surrounding wall thickness, or 0.7 times the surrounding wall thickness, or 0.5 times the surrounding wall thickness, or 0.3 times the surrounding wall thickness, or less.

According to at least one embodiment, the described method enables to manufacture a thinner window thickness than the conventional method. It also enables to integrate the windows to the adapter body with no seams. The manufacturing method of the adapter can therefore be single shot injection molding which is a fast and reliable and also cost efficient way to produce high volumes of products effectively and with good yield. Material properties will affect this method as well, but the window thickness can be less than half of the thickness achieved by conventional methods. The thinner windows increase transmission considerably for the measurement path. The design also enables to produce constant window to window distance, as the product comes from the mold and no assembly is needed. One advantage for at least one embodiment disclosed is that if the windows are integrated into a larger part, that part has a lot more volume and therefore the molten resin, for example plastic, takes more time to cool down than a smaller part with less material volume. This enables the pin to move more easily as the material does not cool down immediately after injection. This is contrary to what would happen in a small thin walled part. If the windows are integrated into a bigger part, there is also more room for the pressed material to flow away from the window area. The moving pins enable the confidence of fill under the pins before the pins are pushed to their final positions.

According to one embodiment, each window has one moving pin. According to one embodiment, each of the pins move a distance less than 1 mm, preferably less than 0.4 mm, preferably less than 0.2 mm. According to one embodiment, each of the pins reduce the final window thickness to less than 0.2 mm, preferably less than 0.15 mm, preferably less than 0.1 mm, preferably less than 0.05 mm. This movement may be done when the pin is in contact with the resin in the mold. According to at least one embodiment, the relationship between the initial thickness and the final thickness is about thirty to one, twenty to one, or fifteen to one, or less. According to one embodiment, a wall thickness surrounding the windows is 30 times, preferably 20 times, preferably 15 times that of the windows thickness 32. The final window thickness 32 may be less than 0.2 mm, preferably less than 0.15 mm more preferably less than 0.1 mm and optimally around 0.05 mm.

According to one embodiment, the airway adapter is molded with two windows opposite each other. According to another embodiment, the airway adapter may have four windows, preferably two pairs of windows, each pair having two windows opposite each other.

According to one embodiment, the relationship between the initial thickness and the finale thickness 32 of the window 30 is about thirty to one, twenty to one, or fifteen to one, or less. The initial thickness of the window is the thickness the window, the thickness of the resin under the pin 200 in the mold, before the pin starts to move. The final thickness 32 is the thickness of the window when the airway adapter is ready, comes out of the mold. The final thickness is the thickness of the resin under the pin 200 in the mold when the pin has finished moving, pressing the window. According to one embodiment, a wall thickness surrounding the windows may be 30 times, preferably 20 times, preferably 15 times that of the windows thickness 32.

According to one embodiment, polypropylene, PP, and/or polyethylene, PE, is used for molding the airway adapter. The resin is preferably a plastic with good flow properties such as PP and/or PE. Other available polymers may be used.

According to one embodiment, the pins dimensions decide the final size of the windows. The shape of the pin determines the area of the window, while the movement of the pin determines the thickness of the window. In this way the whole volume of the window can be determined. Pin movement can be limited for example by having a shoulder feature on the pin that restricts the pin movement to the desired dimensional value regardless on the force that is applied to the pins.

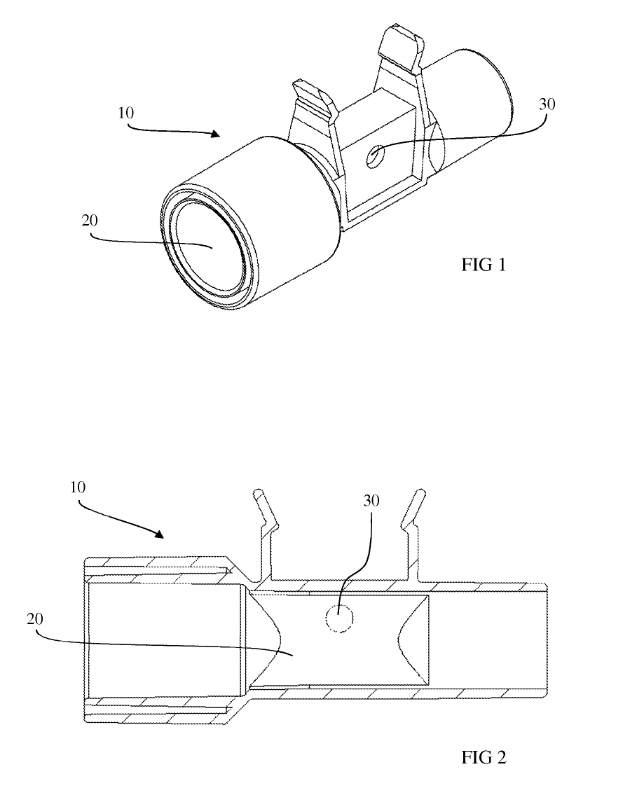

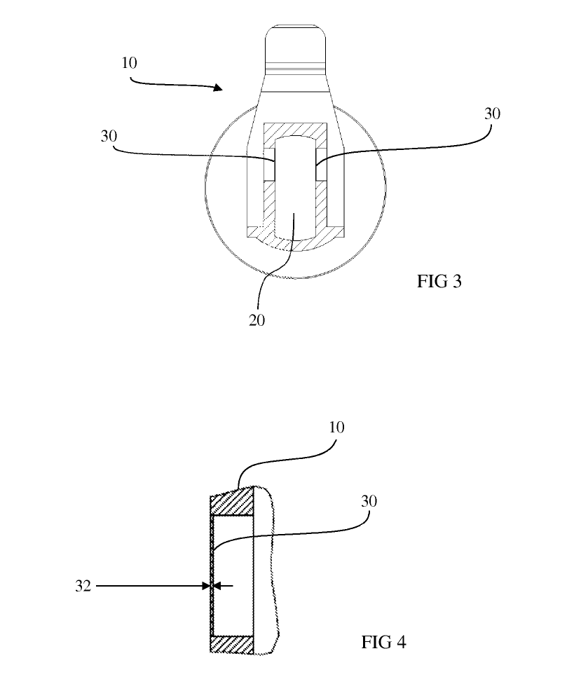

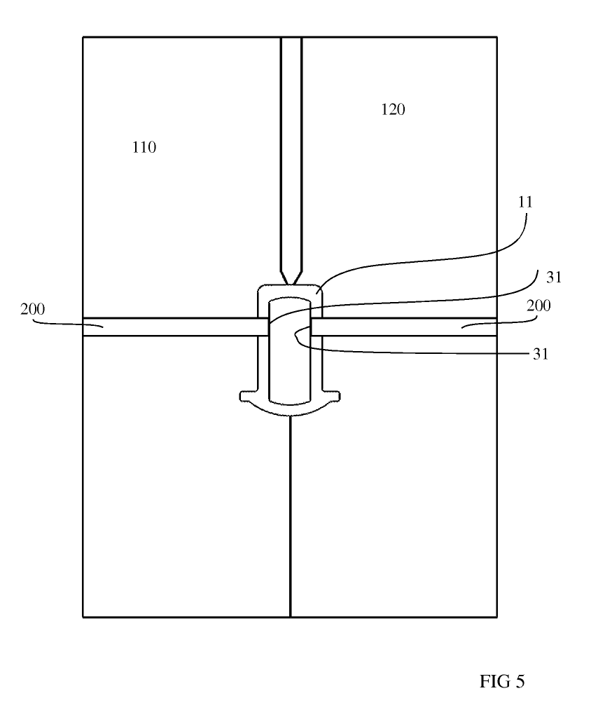

According to one embodiment, an airway adapter 10 may comprise a flow path 20 for fluid and windows 30 for a measurement path through the flow path 20. The windows 30 may be located on opposite sides of the airway adapter 10. The windows 30 may be integral with the airway adapter 10 forming one solid part of the airway adapter joint-less and continuously. The windows may have a thickness 32 of less than 0.2 mm, preferably less than 0.15 mm more preferably less than 0.1 mm, and preferably around 0.05 mm. An embodiment of the airway adapter 10 is illustrated in FIGS. 1-4. What has been described throughout the whole description with regard to the method of making the airway adapter also describes the airway adapter itself, as the product deriving directly from the method for molding the airway adapter.

According to one embodiment, the window and the airway adapter is made of one and the same material. The airway adapter may, for example, be made entirely out of polypropylene, PP, and/or polyethylene, PE. According to one embodiment, a wall thickness surrounding the windows is 30 times, preferably 20 times, preferably 15 times that of the windows thickness 32.

According to at least one embodiments, the size and diameter of the windows 30 may be dependent on the sensor dimensions. A window with a smaller diameter may be advantageous to manufacture because there is less material to move during the pressing with the pins. There is also less force needed if the pin head area is smaller. According to one embodiment, the diameter of the window, and the pin, may be less than 10 mm, or preferably the diameter would be less than 7 mm, or preferably the diameter would be less than 5 mm. However, the diameter of the pin, and the windows, may be more than 2 mm, preferably 3 or 4 mm. The shape of the head of the pin 200 head may comprise a rounded edge. The pin 200 may be of a round shape, but another shape may be used, for example square, rectangle, triangle, or a polygon. The head of the pin 200 may have a small rounded edge. This makes the material flow more easily to and from the window area. It also makes the structure more durable as there are smaller stress condensations around the window edge area. By shaping the head of the pin 200 a lens effect can be easily be manufactured for the windows 30. A head with a convex or concave shape would press and mold the corresponding window with a shape of a lens. A head with a flat shape would press and mold a window free of any lens effect. A window with a lens effect or with no lens effect can advantageously be manufactured according to at least one embodiment.

According to at least one embodiment, any injection moldable material, resin, may be used. Most suitable are the ones that have good flow characteristics and low price, like polypropylene and/or polyethylene. It has been discovered that this material can be used by the disclosed embodiments to mold windows that are acceptable for making the measurement.

According to at least one embodiment, benefits and technical effects include one or more of the following: no backstep creating problems with moisture; no leaks from weld/glue seams between different parts; easy to manufacture; inexpensive to manufacture; no manual assembly work; PP and other semi-crystalline materials can be used without the drawback of difficult welding/heatsealing; dimensional stability between the adapters, where the dimensions coming from the mold remains; no stacking tolerances like in assemblies. At least one embodiment provides as a technical effect that a very inexpensive complete airway adapter can be manufactured in a short time and of a very good and consistent quality, for example no leakage and high transmission properties and with consistent window to window distance.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

What is claimed is:

1. A method for molding an airway adapter, the airway adapter comprising a flow path for fluid and at least two integral windows to allow a measurement path to pass through the flow path, the method comprising:
injection molding the airway adapter in a single shot molding step,
wherein during the single shot molding step, the windows are initially thicker than their final thickness and reduced to their final thickness by moving pins that press the windows to their final thickness.

2. The method according to claim 1, wherein each window has one moving pin.

3. The method according to claim 1, wherein the pins reduce the final window thickness to less than 0.2 mm.

4. The method according to claim 1, wherein the pins reduce the final window thickness to less than 0.1 mm.

5. The method according to claim 1, wherein the pins reduce the final window thickness to less than 0.05 mm.

6. The method according to claim 1, wherein the airway adapter is molded with two windows opposite each other.

7. The method according to claim 1, wherein the relationship between the initial thickness and the finale thickness is about thirty to one, or less.

8. The method according to claim 1, wherein polypropylene or polyethylene is used for molding the airway adapter.

9. The method according to claim 1, wherein the pins dimensions decide the final size of the windows.

10. An airway adapter comprising,
a flow path for fluid;
at least two windows for a measurement path through the flow path, the windows being located on opposite sides of the airway adapter;
the windows being integral with the airway adapter forming one solid part of the airway adapter jointlessly and continuously; and
the windows having a thickness of less than 0.2 mm.

11. The airway adapter according to claim 10, wherein the window has a thickness of less than 0.15 mm.

12. The airway adapter according to claim 10, wherein the window has a thickness of less than 0.1 mm.

13. The airway adapter according to claim 10, wherein the window has a thickness of less than 0.05 mm.

14. The airway adapter according to claim 10, wherein the window and the airway adapter is made of one and the same material.

15. The airway adapter according to claim 10, wherein the airway adapter is made entirely out of polypropylene or polyethylene.
